(19) United States
(12) Patent Application Publication
(10) Pub. No.: US 2026/0108380 A1
(43) Pub. Date: Apr. 23, 2026

(54) ORAL DEVICE WITH PROTRUSIONS

(71) Applicant: DCSTAR INC, New York, NY (US)

(72) Inventor: David LUO, New York, NY (US)

(73) Assignee: DCSTAR INC, New York, NY (US)

(21) Appl. No.: 18/924,157

(22) Filed: Oct. 23, 2024

(51) Int. Cl. A61F 5/56

(52) U.S. Cl. CPC A61F 5/566

(57) ABSTRACT

An oral device that includes an upper tray assembly and a lower tray assembly. The upper tray assembly includes an upper moldable component and an upper bracket, while the lower tray assembly includes a lower moldable component and a lower bracket. Additionally, the oral device has an end point positioned away from the lip side when the patient uses it. An advantage of this disclosure is that the upper bracket has at least one downward-facing lower protrusion near the end point, while the lower bracket includes at least one upward-facing upper protrusion near the end point. The disclosure meticulously defines various parameters and positions for the upper and lower protrusions, ensuring the oral device is better suited to meet the patient's needs.

### TECHNICAL FIELD

This disclosure relates to an oral device equipped with upper and lower protrusions, which enhances convenience and comfort while being cost-effective.

### BACKGROUND

Respiratory-related diseases are common health issues in modern society, including chronic obstructive pulmonary disease (COPD), asthma, pneumonia, tuberculosis, and sleep apnea syndromes such as Obstructive Sleep Apnea (OSA). Severe respiratory diseases can significantly affect patients'quality of life and health, such as Sleep Apnea Syndrome. Sleep Apnea Syndrome is a prevalent sleep disorder characterized by repeated episodes of breathing cessation and hypoventilation during sleep. It includes Obstructive Sleep Apnea Hypopnea Syndrome (OSAHS), Central Sleep Apnea (CSA) due to central nervous system anomalies, and Mixed Sleep Apnea Syndrome (MSAS) combining symptoms of both types. Each type of sleep apnea has specific symptoms, causes, and treatment options, with Obstructive Sleep Apnea being the most common form.

Different respiratory diseases may require different treatment approaches. Current treatments for sleep apnea include medication, lifestyle changes, respiratory therapy, physical therapy, and surgical interventions in special cases. Early diagnosis and treatment of respiratory diseases are crucial due to the high potential for interrelated conditions within the respiratory system.

Among respiratory-related diseases, the primary treatments for the common and severe condition of obstructive sleep apnea mainly include Continuous Positive Airway Pressure (CPAP) therapy, oral appliance therapy (Mandibular Advancement Devices or MADs), upper airway stimulation, lifestyle changes, and surgery. CPAP therapy is generally the first choice for patients with OSA, where the treatment involves covering at least one of the patient's airways with a mask or nasal mask to deliver continuous high-pressure air, preventing airway collapse and obstruction during sleep to maintain normal breathing. For patients who do not tolerate CPAP therapy or those with mild to moderate OSA, oral appliances are a more suitable option due to their compactness and portability. They work by moving the jaw and tongue forward to increase the space in the upper airway, reducing airway obstruction. Additionally, upper airway stimulation and surgery are viable options for more severe cases of OSA. Compared to CPAP machines, oral appliances are easier to adapt to and wear, making them especially convenient for patients who travel frequently as they require no power supply and are more compact and portable, thus improving treatment compliance. CPAP therapy can sometimes cause side effects such as nasal dryness, congestion, throat discomfort, and skin irritation on the face, whereas oral appliances typically produce fewer of these side effects, making them a favorable alternative to CPAP. Moreover, oral appliances do not generate noise, offering a more economical option for patients with limited financial resources.

When choosing a treatment plan, it is important to consider the severity of symptoms, budget, and personal needs to find the most suitable treatment approach.

### SUMMARY

The present disclosure addresses the shortcomings mentioned above by providing an oral device that is convenient for patients to use, reduces costs for the patient, and offers comfortable wear.

An oral device with protrusions is provided. The oral device includes an upper tray assembly, including an upper moldable component and an upper bracket, and having an arcuate outer shape configured to conform to a curvature of a patient's upper dental arch. The oral device further includes a lower tray assembly, including a lower moldable component and a lower bracket, and having an arcuate outer shape configured to conform to a curvature of the patient's lower dental arch. The upper bracket and the lower bracket each are formed by an outer wall and a bottom wall, each having two opposite sides configured to be near cheeks when in use. The oral device has an end point configured to be positioned away from a lip side. The upper moldable component is configured to be in connection with at least the bottom wall of the upper bracket. The lower moldable component is configured to be in connection with at least the bottom wall of the lower bracket. A temperature required to heat the upper moldable component and the lower moldable component to deform is different from that required for the upper bracket and the lower bracket. The upper bracket and the lower bracket each have at least one side of the two opposite sides near the cheeks that includes an upper protrusion and a lower protrusion, respectively. The upper protrusion and the lower protrusion are configured to allow the patient's mouth to open and close while maintaining a fixed anterior-posterior positional relationship between the upper bracket and the lower bracket when in use. The upper protrusion has a first side nearer the end point and a second side opposite the first side. The lower protrusion has a third side nearer the end point and a fourth side opposite the third side. The second side and the third side are configured to be in contact when in use to limit a distance of an anterior displacement of the lower bracket. The upper protrusion and the lower protrusion each have at least a portion configured to be positioned nearer the cheeks than the outer walls of the upper bracket and the lower bracket. A distance from the upper protrusion to the end point of the oral device is shorter than a distance from the lower protrusion to the end point of the oral device.

In one embodiment, the arcuate outer shapes of the upper tray assembly and the lower tray assembly adopt at least one of an ovoid arch, a tapered arch, or a square arch.

In one embodiment, a material of the upper moldable component and the lower moldable component includes a flexible thermoplastic material.

In one embodiment, the upper moldable component and the lower moldable component are configured to soften when heated in hot water, allowing them to be adjusted and molded to match the curvatures of the patient's upper dental arch and lower dental arch during biting.

In one embodiment, a hardness of a material of the upper bracket and the lower bracket is at least partially greater than a hardness of the material of the upper moldable component and the lower moldable component.

In one embodiment, the connection between the upper moldable component and the bottom wall of the upper bracket, and between the lower moldable component and the bottom wall of the lower bracket, is established through one of the following methods: snap-fit connection, adhesive bonding, or groove connection.

In another embodiment, an oral device with protrusions is provided. The oral device includes an upper tray assembly, including an upper moldable component and an upper bracket, configured to be positioned adjacent to a patient's upper dental arch when placed inside the patient's mouth. The oral device further includes a lower tray assembly, including a lower moldable component and a lower bracket, configured to be positioned adjacent to the patient's lower dental arch when placed inside the patient's mouth. The upper bracket and the lower bracket each are formed by an outer wall and a bottom wall, each having two opposite sides configured to be near cheeks when in use. The oral device has an end point configured to be positioned away from a lip side. The upper moldable component is configured to be in connection with at least the bottom wall of the upper bracket. The lower moldable component is configured to be in connection with at least the bottom wall of the lower bracket. A temperature required to heat the upper moldable component and the lower moldable component to deform is different from that required for the upper bracket and the lower bracket. The upper bracket and the lower bracket each have at least one side of the two opposite sides near the cheeks that includes an upper protrusion and a lower protrusion, respectively. The upper protrusion and the lower protrusion are configured to allow the patient's mouth to open and close while maintaining a fixed anterior-posterior positional relationship between the upper bracket and the lower bracket when in use. The upper protrusion has a first side nearer the end point and a second side opposite the first side, and the lower protrusion has a third side nearer the end point and a fourth side opposite the third side. The second side and the third side are configured to be in contact when in use to limit a distance of an anterior displacement of the lower bracket, and the second side and/or the third side is curved. A distance from the upper protrusion to the end point of the oral device is shorter than a distance from the lower protrusion to the end point of the oral device.

In one embodiment, a distance between at least a portion of the upper protrusion and the outer wall of the upper bracket is greater than or equal to a distance between the lower protrusion and the outer wall of the lower bracket.

In one embodiment, the connection between the upper moldable component and the upper bracket, and between the lower moldable component and the lower bracket, is established through grooves formed by the outer walls and the bottom walls of the upper bracket and the lower bracket, respectively.

In one embodiment, the upper protrusion and the lower protrusion have shapes that conform to curvatures of the outer walls of the upper bracket and the lower bracket, respectively.

In one embodiment, the upper moldable component and the lower moldable component include a material with plasticity, and the temperature required to heat the upper moldable component and the lower moldable component to deform is less than 100 degrees Celsius.

In one embodiment, the upper bracket and the lower bracket include a material with high thermal stability, and the temperature required to heat the upper bracket and the lower bracket to deform is greater than 100 degrees Celsius.

In yet another embodiment, an oral device with protrusions is provided. The oral device includes an upper tray assembly, including an upper moldable component and an upper bracket, and having an arcuate outer shape configured to conform to a curvature of a patient's upper dental arch. The oral device further includes a lower tray assembly, including a lower moldable component and a lower bracket, and having an arcuate outer shape configured to conform to a curvature of the patient's lower dental arch. The upper bracket and the lower bracket each are formed by an outer wall and a bottom wall, each having two opposite sides configured to be near cheeks when in use. The oral device has an end point configured to be positioned away from a lip side. The upper moldable component is configured to be in connection with at least the bottom wall of the upper bracket. The lower moldable component is configured to be in connection with at least the bottom wall of the lower bracket. A temperature required to heat the upper moldable component and the lower moldable component to deform is different from that required for the upper bracket and the lower bracket. The upper bracket and the lower bracket each have at least one side of the two opposite sides near the cheeks that includes an upper protrusion and a lower protrusion, respectively. The upper protrusion and the lower protrusion are configured to allow the patient's mouth to open and close while maintaining a fixed anterior-posterior positional relationship between the upper bracket and the lower bracket when in use. The upper protrusion has a first side nearer the end point and a second side opposite the first side. The lower protrusion has a third side nearer the end point and a fourth side opposite the third side. The second side and the third side are configured to be in contact when in use to limit a distance of an anterior displacement of the lower bracket. The upper protrusion and the lower protrusion have one or more of the following characteristics: a. a height of the upper protrusion and the lower protrusion being between 1.7 mm to 22.5 mm; b. a distance from the upper protrusion and the lower protrusion to the outer walls of the upper bracket and the lower bracket, respectively, being less than 17.5 mm.

In one embodiment, the upper tray assembly and the lower tray assembly are shaped such that they are higher in the middle and lower on both sides.

In one embodiment, a projected area of the upper moldable component and the lower moldable component on a horizontal plane is greater than a projected area of the upper bracket and the lower bracket on the same plane.

In one embodiment, the outer walls of the upper bracket and the lower bracket have a continuous curvature.

In one embodiment, a connection point of the upper protrusion and the upper bracket, and/or of the lower protrusion and the lower bracket, is flush with the bottom walls of the upper bracket and the lower bracket.

Another form of the oral device with protrusions is also provided. The oral device includes an upper tray assembly, including an upper moldable component and an upper bracket, configured to be positioned adjacent to a patient's upper dental arch when placed inside the patient's mouth. The oral device further includes a lower tray assembly, including a lower moldable component and a lower bracket, configured to be positioned adjacent to the patient's lower dental arch when placed inside the patient's mouth. The upper bracket and the lower bracket each are formed by an outer wall and a bottom wall, each having two opposite sides configured to be near cheeks when in use. The oral device has an end point configured to be positioned away from a lip side. The upper moldable component is configured to be in connection with at least the bottom wall of the upper bracket. The lower moldable component is configured to be in connection with at least the bottom wall of the lower bracket. A temperature required to heat the upper moldable component and the lower moldable component to deform is different from that required for the upper bracket and the lower bracket. The upper bracket and the lower bracket each have at least one side of the two opposite sides near the cheeks that includes an upper protrusion and a lower protrusion, respectively. The upper protrusion and the lower protrusion are configured to allow the patient's mouth to open and close while maintaining a fixed anterior-posterior positional relationship between the upper bracket and the lower bracket when in use. The upper protrusion has a first side nearer the end point and a second side opposite the first side. The lower protrusion has a third side nearer the end point and a fourth side opposite the third side. The second side and the third side are configured to be in contact when in use to limit a distance of an anterior displacement of the lower bracket. The upper bracket and the lower bracket have a front end near the lip side when in use, and a distance from the upper protrusion and/or lower protrusion to the front end is greater than 10 mm.

In one embodiment, a width of the upper protrusion and the lower protrusion is greater than 2.5 mm.

In one embodiment, the upper tray assembly and the lower tray assembly include at least two different materials.

In one embodiment, a projected area of the upper moldable component and the lower moldable component on a horizontal plane is greater than a projected area of the upper bracket and the lower bracket on the same plane.

In one embodiment, the upper protrusion and the lower protrusion form an angle with the outer walls of the upper bracket and the lower bracket, respectively.

The oral appliance implemented by this disclosure offers several beneficial effects:

1) Oral appliances on the market are primarily divided into two categories: customized and non-customized. Customized oral appliances are used to fit a patient's teeth through scanning, ensuring a perfect match with the patient's dental and oral conditions. This customization provides better comfort and stability, allowing the appliance to remain stable even when the mouth is open. However, they require customization under professional medical guidance and the use of high-precision and digital equipment, making them more expensive. Typically, a set of customized mandibular advancement devices can cost patients thousands of dollars and involve complex and cumbersome customization processes that require ongoing adjustments and coordination with a physician. On the other hand, non-customized oral appliances do not require dental scanning for customization, making them a more economical treatment option. They are also relatively easy to use; they simply need to be heated and molded to fit the upper and lower dental arches. However, because they are self-molded by the patient after heating, their fit and stability are not as good as those of customized appliances. Furthermore, user feedback collected from various e-commerce platforms indicates generally poor reviews for existing non-customized mandibular advancement devices. Based on the conclusions above, this disclosure innovatively combines the advantages of both customized and non-customized oral devices, enhancing stability, therapeutic effectiveness, and user comfort while maintaining economic benefits. The device is designed to remain effective even when the mouth opens and closes. This new oral device offers both economic and functional advantages, providing patients with a better option.

2) The oral appliance with upper and lower protrusions offers functional advantages. Traditional moldable oral appliances are fixed structures that limit oral movement when worn. In contrast, this disclosure's appliance with upper and lower protrusions allows patients to speak, drink, and perform other daily activities while wearing it, greatly expanding its range of application. This design ensures that patients do not need to frequently remove the appliance during treatment, enhancing user convenience. This convenience is particularly important for patients who need to wear oral appliances for extended periods, significantly improving their quality of life. Specifically, for some patients who experience throat snoring symptoms, these individuals may need to breathe through their mouths while sleeping, a situation that traditional moldable oral appliances struggle to accommodate. The protrusion design of this oral appliance can adapt to various oral movements, thus offering more effective treatment for a broader patient group. This multifunctional design meets the personalized needs of different patients.

3) The design and development of the oral appliance with upper and lower protrusions is not just about structure; it involves a complex and rigorous process that incorporates multiple types of data for design, testing, and experimentation. Initially, it requires gathering oral data from different patient groups to ensure that the device meets the needs of various oral structures. Subsequently, the collected data is used for preliminary design and simulation, material selection and testing, numerous experimental tests and optimizations, and population trials and validations to ensure the appliance's functionality and comfort under diverse conditions. Finally, the optimal structure of the oral appliance is determined, including the form and placement of the upper and lower protrusions, material thickness, and the angles, height, and length of the structure. Each step is meticulously calculated and repeatedly validated, ensuring the product achieves the best possible functionality, comfort, and safety, while providing an effective and comfortable treatment experience for patients.

4) Dentistry is a precise and data-driven discipline where even minor variations in data (such as angles, weight, dimensions, and the softness of materials) can impact patient comfort and the effectiveness of use. This disclosure meticulously adjusts and repeatedly tests these variables to achieve optimal usage and comfort levels. Further in-depth research into the design of oral appliances has addressed numerous challenges, enhancing comfort during treatment. Since oral appliances must conform closely to the human body, they require extensive consideration of human factors engineering and are designed and tested using substantial human data, making them complex and challenging technologies when designed and manufactured. Specifically, during the process of opening the mouth to wear the appliance, it must counteract its own weight to maintain stable engagement with the upper dental arch of the patient; the heights and widths of the upper and lower protrusions are constrained to allow patients to adapt to the prescribed opening angles while minimizing discomfort when worn; and the design of the upper and lower protrusions ensures that they consistently restrict the lower jaw during mouth opening and closing, maintaining the connection between the upper and lower brackets. Solutions to these issues have been meticulously designed and extensively tested. This includes optimizing the details of the upper and lower brackets and the upper and lower moldable components to better fit the upper and lower dental arches. In particular, the upper tray assembly is closely matched to the upper dental arch to counteract gravity when the mouth is open, ensuring stability; the height range of the upper and lower protrusions is set between 1.7 mm and 22.5 mm to allow for a greater range of mouth opening and closing angles without conflicting with the patient's oral structure. The distances from the upper and lower protrusions to the front end are set to be greater than 10 mm. While positioning the upper and lower protrusions closer to the end point increases the opening angle between the upper and lower brackets, it can also lead to more frequent contact with the patient's oral structures, potentially causing a foreign body sensation. Therefore, finding a balance between these factors is essential. The upper and lower brackets need the upper and lower protrusions to engage at any angle within a certain range, so positioning the upper and lower protrusions outward at a certain distance is crucial to ensure therapeutic effectiveness and enhance patient comfort. Furthermore, it's important to design at least one curvature at the contact points of the upper and lower protrusions of the upper and lower brackets. This curvature ensures smooth operation of the brackets during opening and closing, reducing friction and the possibility of getting stuck. These designs are not merely simple structural forms but result from precise calculations and optimizations. To achieve the best outcomes, numerous experimental tests and data recordings are necessary. Through repeated trials and minor adjustments, the shapes and metrics of the upper and lower protrusions are gradually refined. Extensive data analysis and feedback are used to optimize the design concerning the upper and lower protrusions and other aspects, ensuring that every detail undergoes rigorous verification and optimization. This meticulous approach guarantees the comfort of the patients during use and the durability of the oral appliance.

5) The oral appliance of this disclosure also has the following advantages: a. The oral appliance with upper and lower protrusions enhances treatment stability. The structure of the upper and lower protrusions provides a reliable fixation mechanism, reducing movement of the oral appliance and the risk of dislodgement within the oral cavity. This ensures that the oral appliance maintains its effective therapeutic impact in various jaw positions, minimizing the risk of treatment interruptions and improving both the effectiveness and stability of the product. Additionally, the enhanced stability of this disclosure's oral appliance means that patients do not need to replace it frequently, reducing subsequent replacement costs. The fixed structure of the upper and lower protrusions requires patients to purchase a set of upper and lower tray assemblies to ensure the appropriate mandibular advancement distance. While this increases the initial cost to the patient, the fixed protrusions are more stable and durable compared to adjustable structures, reducing abandonment due to damage and further lowering the overall treatment costs. b. Unlike conventional customized mandibular advancement devices, whose main body (i.e., the part that directly contacts the teeth) is typically made from a rigid material, this disclosure's oral device features flexible materials that contact the inner mouth. These materials do not irritate the patient's oral tissues, reducing the foreign body sensation when wearing the product. Additionally, in the event of an accidental impact, the flexible materials act as a cushion, protecting the teeth and offering a safer oral device. c. This disclosure's oral device can be quickly produced and used, saving patients the waiting time associated with purchasing customized devices. This makes it a superior option for patients seeking prompt treatment.

### BRIEF DESCRIPTION OF THE DRAWINGS

FIG. 1 is an overview diagram of the human respiratory system, including the nasal cavity and oral cavity, larynx, esophagus, trachea, and bronchi;

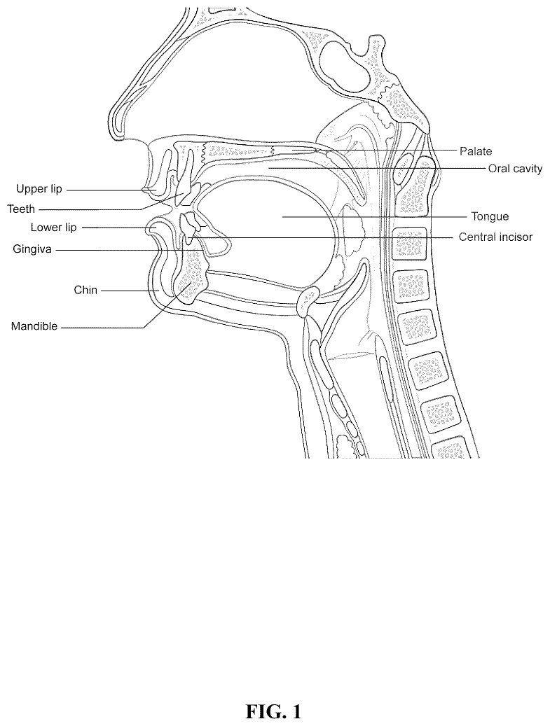

FIG. 2 is an anatomical overview diagram of the human oropharynx, featuring several identified surface anatomical structures, including the oral cavity, pharynx, cervical vertebrae, oral glands, and mandible;

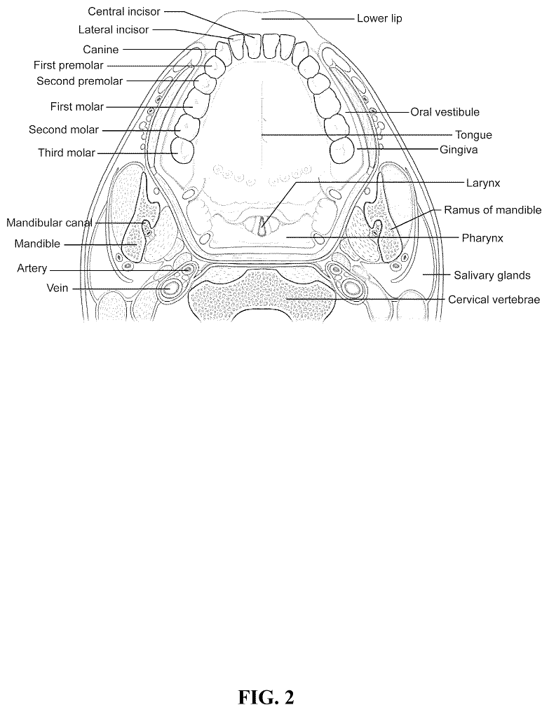

FIG. 3 is an overview diagram of the interior of the human oral cavity, featuring several identified surface anatomical structures, including the teeth, palate, pharynx, tongue, and gums;

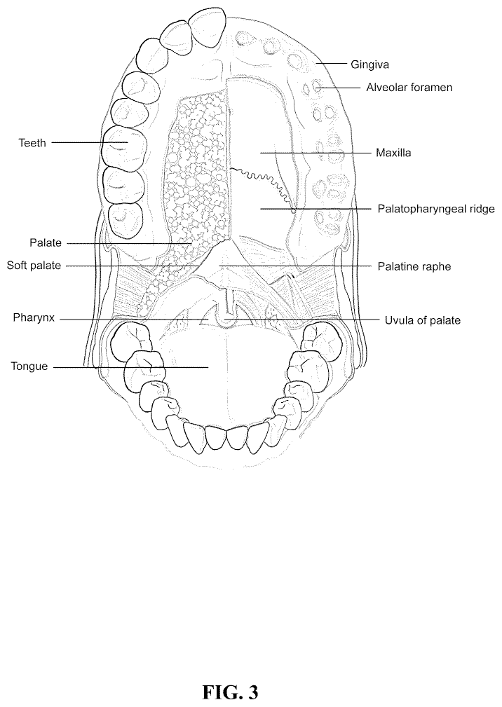

FIG. 4 is a side view of the human skull, including the mandible, teeth, spine, larynx, maxilla, and condyle.

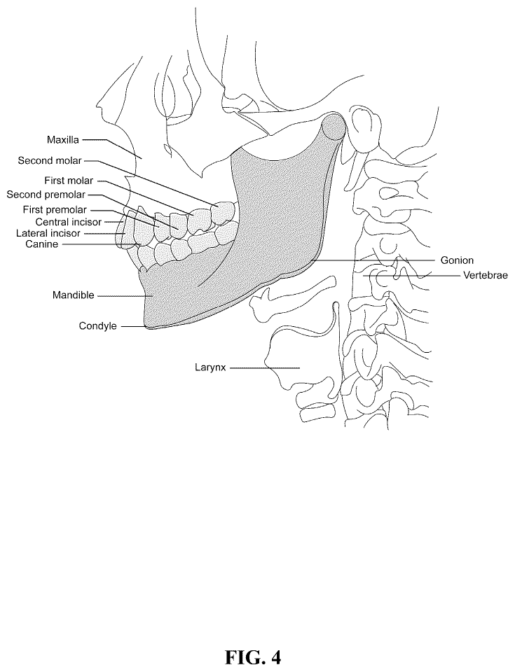

FIG. 5 is a side view of the human skull and oral muscles, including the buccinator muscle, orbicularis oris muscle, lips, and oral commissure;

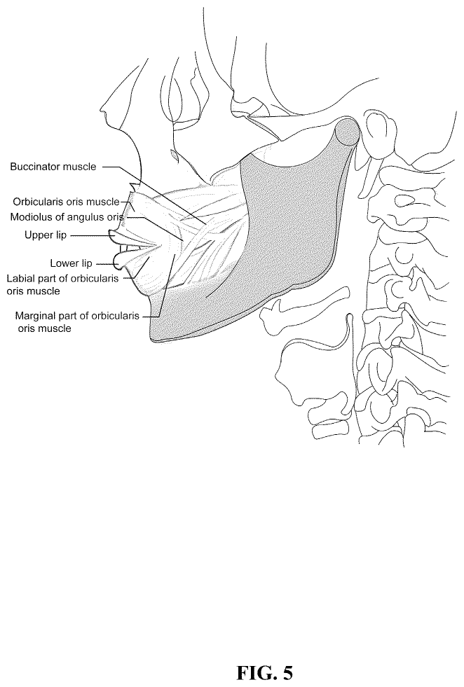

FIG. 6 is a side view of the lower jaw structure in the human head, showing the general positions of the upper teeth and the mandible;

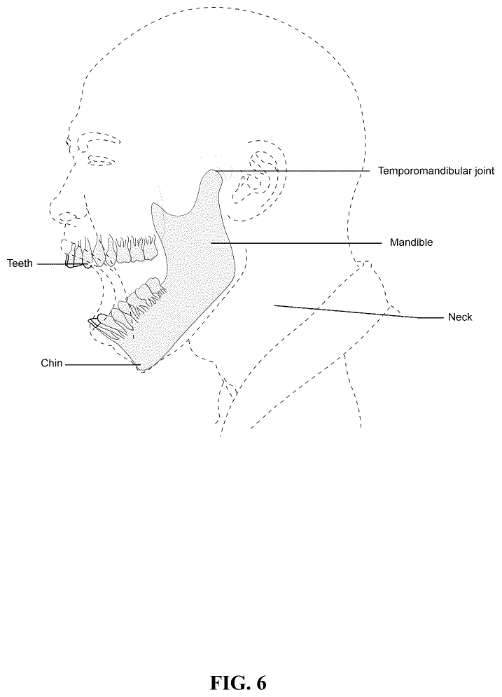

FIG. 7 is a schematic diagram of the specific structure of the mandible, including the teeth, chin, mandibular neck, condylar process, and mandibular head;

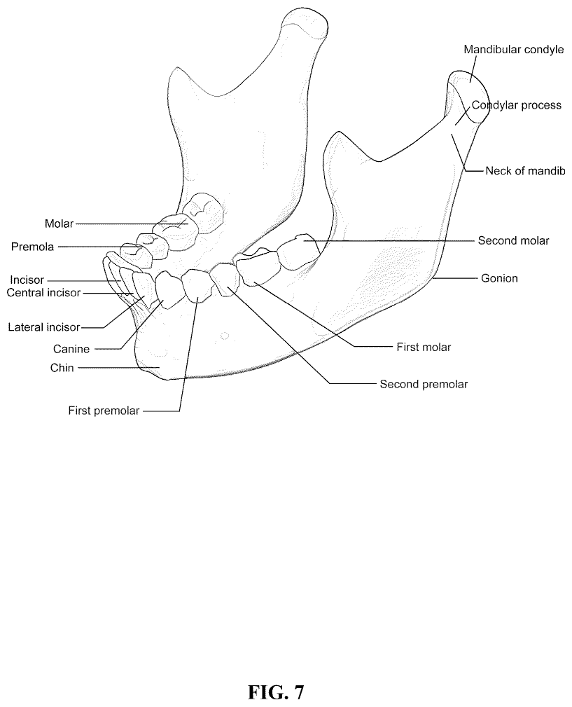

FIG. 8 is an anatomical overview diagram of the mandible, including the teeth, mandibular body, mental spine, and mandibular ramus;

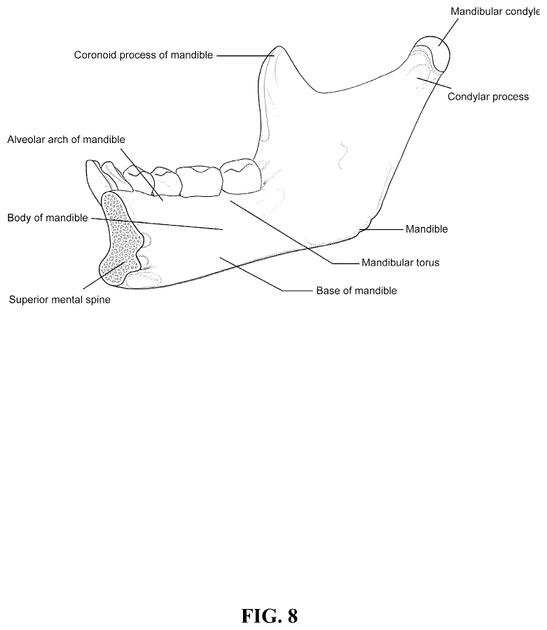

FIG. 9 is a schematic diagram of the movement of the mandible relative to the maxilla, illustrating the rotation and movement of the mandible with the condylar process as a pivot point;

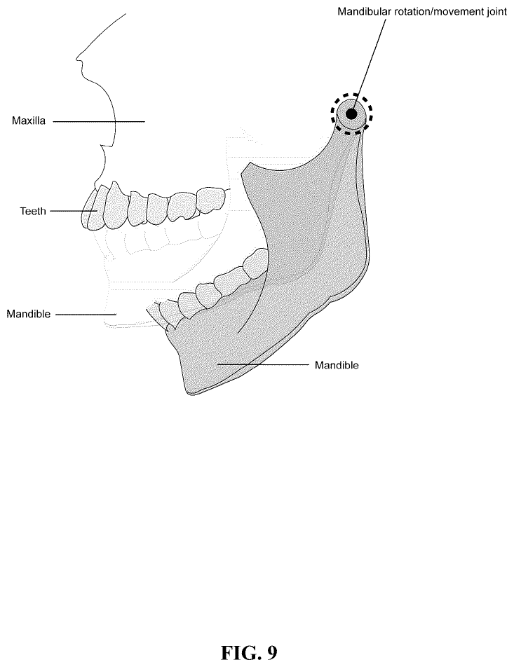

FIG. 10 is a general schematic diagram of an oral device according to an embodiment of the present disclosure;

FIG. 11 is an exploded view of the structure of an oral appliance according to an embodiment of the present disclosure;

FIGS. 12A, 12B, and 12C are top views showing the upper tray assembly and the shape of the upper dental arch according to several embodiments of the present disclosure;

FIG. 13 is a schematic diagram showing the matching connection between the upper moldable component and the upper bracket according to an embodiment of the present disclosure;

FIG. 14 is a three-dimensional schematic diagram of the upper bracket with an upper protrusion and the lower bracket with a lower protrusion according to an embodiment of the present disclosure;

FIG. 15 is a side view of an oral device according to an embodiment of the present disclosure;

FIG. 16 is a three-dimensional schematic diagram of an oral device in a closed position according to an embodiment of the present disclosure;

FIG. 17 is a three-dimensional schematic diagram of an oral device according to an embodiment of the present disclosure;

FIG. 18 is a top view of an oral device according to an embodiment of the present disclosure;

FIG. 19 is a sectional view of the upper bracket along the mid-sagittal plane according to an embodiment of the present disclosure;

FIG. 20 is a schematic diagram showing the opening method of an oral device according to an embodiment of the present disclosure;

FIGS. 21A, 21B, 21C are schematic diagrams showing the connection between the upper protrusion of the upper bracket and the lower protrusion of the lower bracket according to an embodiment of the present disclosure;

FIG. 22 is a schematic diagram showing the connection between the upper protrusion and/or lower protrusion and the upper bracket and/or lower bracket according to an embodiment of the present disclosure;

FIG. 23 is a schematic diagram showing the angled connection between the upper protrusion and/or lower protrusion and the upper bracket and/or lower bracket according to an embodiment of the present disclosure;

FIG. 24 is a top view showing the upper bracket and/or lower bracket according to an embodiment of the present disclosure, where the upper protrusion and/or lower protrusion have the same curvature as the outer walls of the brackets;

FIG. 25 is a top view showing the upper bracket and/or lower bracket according to an embodiment of the present disclosure, where the upper protrusion and/or lower protrusion have a different curvature than the outer walls of the brackets;

FIG. 26 is a schematic diagram showing various data related to the upper bracket and/or lower bracket according to an embodiment of the present disclosure;

FIG. 27 is a schematic diagram showing the position of the upper bracket and/or lower bracket according to an embodiment of the present disclosure;

FIG. 28 is a schematic diagram showing another form of the upper protrusion and lower protrusion according to an embodiment of the present disclosure;

FIG. 29 is a schematic diagram showing another form of an oral appliance including only an upper protrusion or a lower protrusion, according to an embodiment of the present disclosure.

### DETAILED DESCRIPTION

To make the objectives, features, and advantages of the present disclosure more apparent and understandable, the specific embodiments of the present disclosure are described in detail below in conjunction with the accompanying drawings. Specific details are set forth in the following description to facilitate a thorough understanding of the present disclosure. However, the present disclosure can be implemented in many different ways other than those described herein, and those skilled in the art can make similar improvements without departing from the essence of the disclosure. Therefore, the present disclosure is not limited by the specific embodiments disclosed below.

Compared to existing oral appliances on the market, the present disclosure improves the range of oral movement for patients wearing the oral appliance during sleep by designing and using the upper and lower protrusions on the upper and lower brackets, while maintaining the effectiveness of the treatment. This enhancement increases the comfort and ease of use of the oral appliance and, to some extent, improves the effectiveness of the device. It ensures long-lasting and effective treatment during the patient's sleep, achieving optimal performance in terms of effectiveness, comfort, reliability, and lifespan. As a result, the oral appliance of the present disclosure not only improves performance metrics but also provides patients with a more stable, safe, and comfortable user experience.

The present disclosure provides an oral appliance 1 that is convenient for patients to use, enhances the ease of use, and is comfortable to wear.

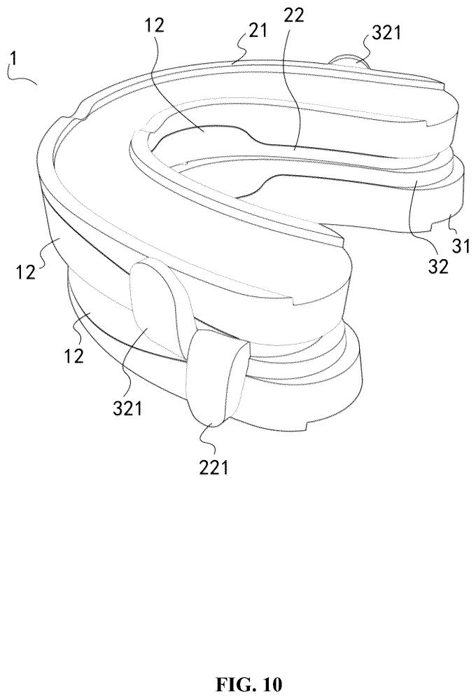

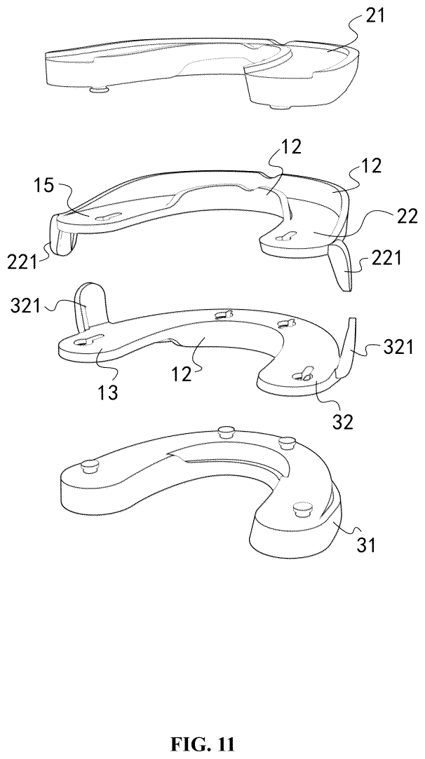

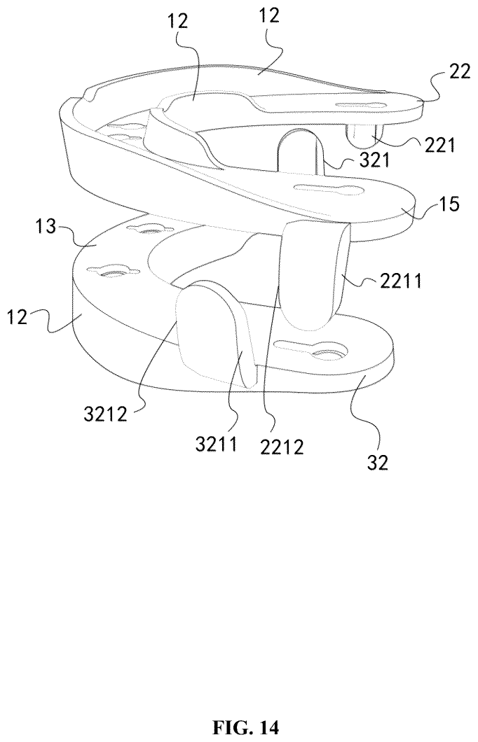

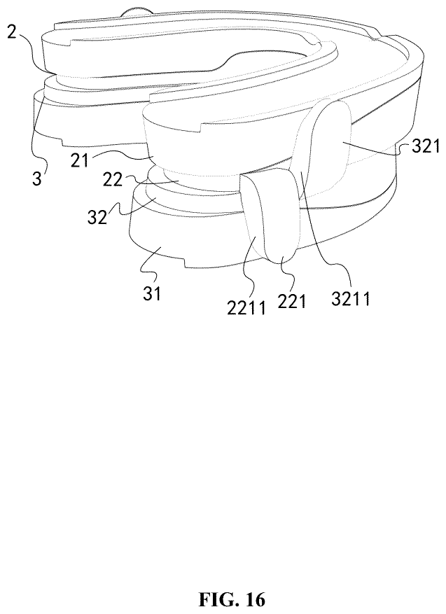

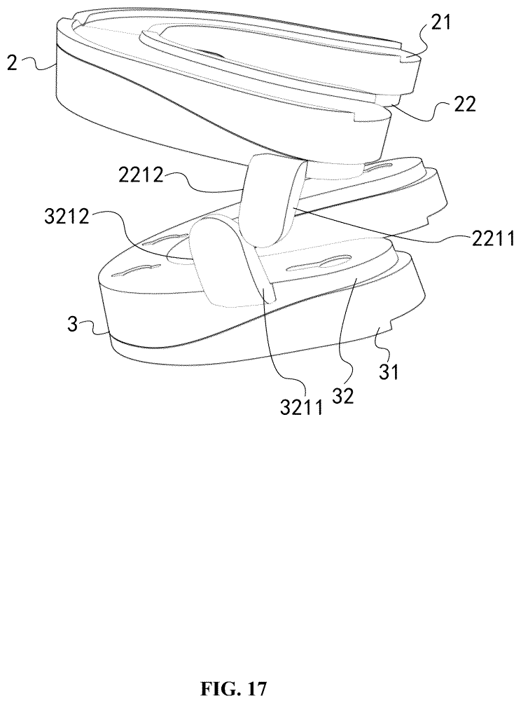

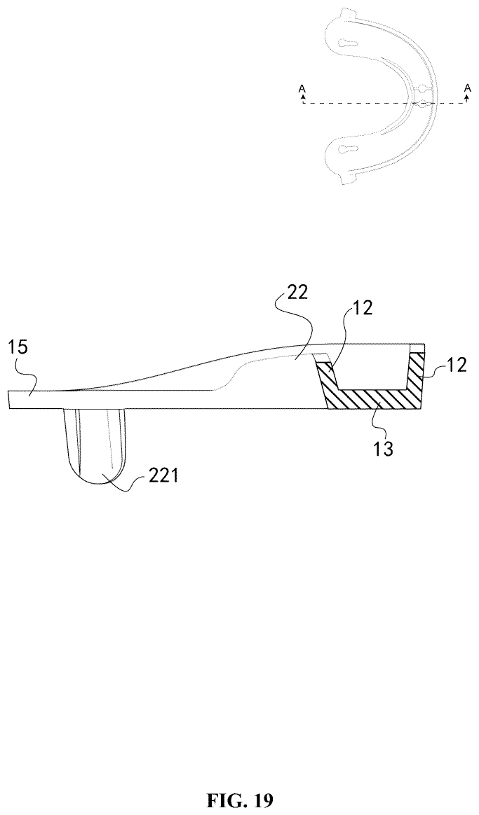

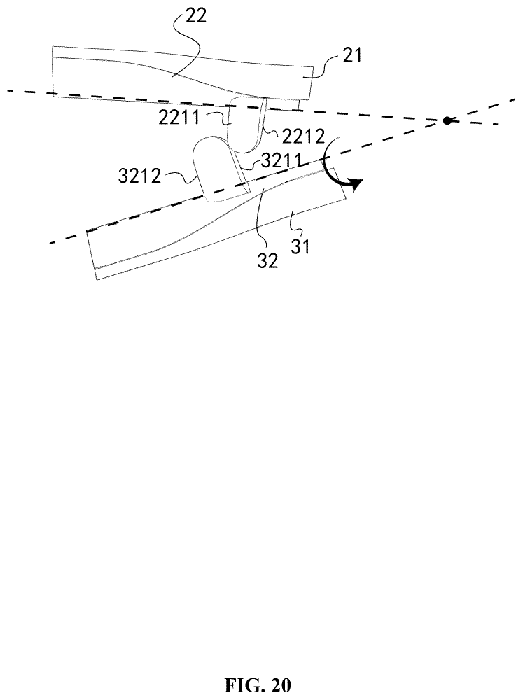

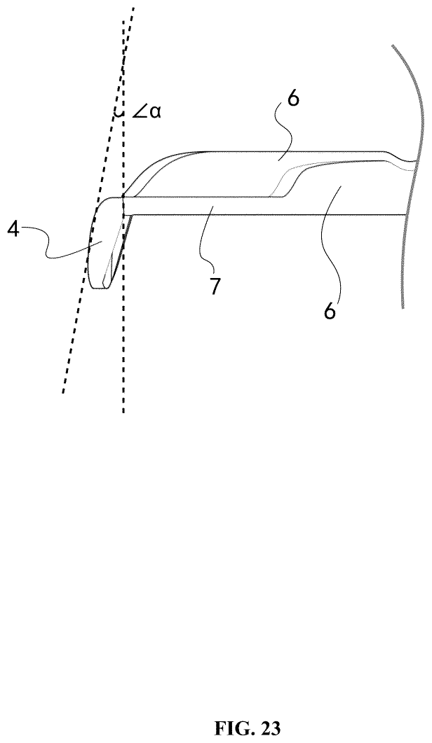

Specifically, referring to FIGS. 10 through 27, the oral appliance 1 of the present disclosure includes an upper tray assembly 2 and a lower tray assembly 3. The upper tray assembly 2 is composed of an upper moldable component 21 and an upper bracket 22, both having an arcuate outer shape to conform to the curvature of the patient's upper dental arch and configured to be positioned adjacent to the patient's upper dental arch when placed inside the mouth. The lower tray assembly 3 is composed of a lower moldable component 31 and a lower bracket 32, both having an arcuate outer shape to conform to the curvature of the patient's lower dental arch and configured to be positioned adjacent to the patient's lower dental arch when placed inside the mouth. To accommodate the internal space of the patient's oral cavity, the upper tray assembly 2 and the lower tray assembly 3 are shaped such that they are higher in the middle (i.e., near the patient's front teeth in the oral device 1) and lower on both sides (i.e., the ends of the oral device 1 near the patient's temporomandibular joint). Typically, the upper tray assembly 2 and the lower tray assembly 3 are separate components. The mandibular advancement effect is achieved through the relative movement of these two components, specifically by moving the lower tray assembly 3 relative to the upper tray assembly 2 towards the lip side and securing it in place. The upper tray assembly 2 is fixed to the patient's upper dental arch, and the lower tray assembly 3 is fixed to the patient's lower dental arch. This arrangement pulls the patient's mandible towards the lip side, thereby achieving a mandibular advancement treatment effect to open the patient's airway.

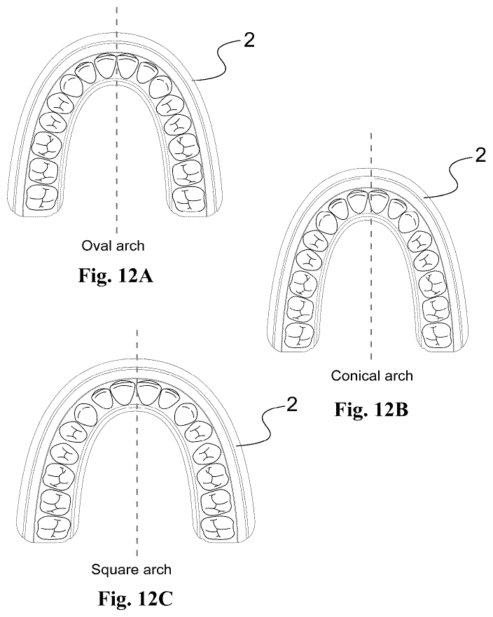

The upper moldable component 21 and the lower moldable component 31 are configured to at least partially contact the patient's upper and lower dental arches when the oral appliance 1 is positioned in the patient's mouth. Both components have surfaces designed to engage with the teeth. These surfaces may feature at least one groove to accommodate overflow material during biting, ensuring a better fit with the patient's upper and lower dental arches. In some cases, the surfaces of the upper moldable component 21 and/or lower moldable component 31 that engage with the teeth may be smooth, without grooves, either flat or curved. The upper moldable component 21 and the lower moldable component 31 are shaped to contact the patient's upper and lower dental arches during wear, matching the natural arcuate structure of the teeth, specifically forming shapes such as ovoid arches, tapered arches, or square arches. Therefore, the arch shapes of the upper moldable component 21 and the lower moldable component 31 adopt at least one of these forms (as shown in FIGS. 12A, 12B, and 12C): ovoid arch, tapered arch, or square arch to ensure full contact with the teeth and gums. To protect the patient's teeth and enhance comfort, the upper moldable component 21 and the lower moldable component 31 are made from a flexible thermoplastic material. This material softens when heated in hot water, facilitating adjustments and molding to match the shapes of the patient's upper and lower dental arches during biting. The hot water used is at least 60 degrees Celsius. Currently, the upper moldable component 21 and the lower moldable component 31 available on the market can be categorized as either custom-made or non-custom-made (i.e., heat-moldable upper moldable component 21 and lower moldable component 31). The production process for heat-moldable components is simpler, only requiring heating and softening to bite and shape to fit the patient's teeth. After setting, they can be reheated for minor shape adjustments. This type of oral appliance 1 can be produced quickly and at a low cost, though its production precision and fit are generally lower than those of custom-made components.

The upper bracket 22 and the lower bracket 32 are designed to engage and support the upper moldable component 21 and the lower moldable component 31, respectively. As such, the hardness of the materials of the upper bracket 22 and the lower bracket 32 is at least partially greater than the hardness of the materials of the upper moldable component 21 and the lower moldable component 31, with the hardness of the materials of the upper bracket 22 and the lower bracket 32 ranging between Shore D45 and Shore D95. The oral appliance 1 pulls the patient's mandible forward. Due to habitual actions, the patient's lower dental arch tends to move back to its original position, exerting a force on the upper tray assembly 3 in a direction away from the lip side, while the upper dental arch exerts force on the upper tray assembly 2 towards the lip side. To ensure that the upper moldable component 21 and the lower moldable component 31 remain securely attached to the upper bracket 22 and the lower bracket 32, respectively, the upper bracket 22 and the lower bracket 32 feature the outer walls 12 that limit the movement of the upper moldable component 21 and the lower moldable component 31. The upper bracket 22 and the lower bracket 32 are comprised of the outer walls 12 and the bottom walls 13, with sides that are near the cheeks when worn, and the oral appliance has an end point 15 that is positioned away from the lip side. Typically, the outer walls 12 of both the upper bracket 22 and the lower bracket 32 have a continuous curvature. The upper bracket 22 and the lower bracket 32 can be connected to the upper moldable component 21 and the lower moldable component 31 in various ways. The connections between the upper moldable component 21 and the lower moldable component 31 with the upper bracket 22 and the lower bracket 32, respectively, can either be detachably engaged or permanently fixed, non-detachable. The detachable engagement methods include protrusions, groove-type snap-fits, and/or additional components such as pins, screws, dowels, and magnets. For enhanced fixation and convenience of the oral appliance 1, it is preferable for the connection between the upper moldable component 21 and the lower moldable component 31 with the upper bracket 22 and the lower bracket 32, respectively, to be a snap-fit connection. This type of snap-fit connection typically involves protrusions on the upper moldable component 21 and the lower moldable component 31 that match grooves on the upper bracket 22 and the lower bracket 32, or vice versa. The protrusions and grooves can be located symmetrically near the middle part (near the patient's front teeth) and/or on the opposite sides and/or at the opposite ends (near the end of the oral appliance 1 towards the patient's mandibular joints). In another configuration, the connection between the upper moldable component 21 and the lower moldable component 31 with the upper bracket 22 and the lower bracket 32, respectively, is formed by the outer walls 12 and bottom walls 13 of the upper bracket 22 and the lower bracket 32 surrounding the upper moldable component 21 and the lower moldable component 31. Regardless of the connection method used, the upper moldable component 21 and the lower moldable component 31 are configured to engage with at least the bottom walls 13 of the upper bracket 22 and the lower bracket 32, respectively.

The fixed connection between the upper tray assembly 2 and the lower tray assembly 3 is typically established through the upper bracket 22 and the lower bracket 32, and this fixed connection can take various forms. Commonly, the connection between the upper bracket 22 and the lower bracket 32 is a snap-fit. However, to ensure that the upper tray assembly 2 and the lower tray assembly 3 remain functional within the patient's mouth while allowing for opening and closing movements of the mouth, in this disclosure, the connection between the upper bracket 22 and the lower bracket 32 is secured through the interaction between an upper protrusion 221 and a lower protrusion 321.

The materials used for the upper tray assembly 2 and the lower tray assembly 3 are chosen from a wide range. Given the different roles played by the upper moldable component 21, the lower moldable component 31, the upper bracket 22, and the lower bracket 32 during treatment, at least two different materials are used in the construction of the upper tray assembly 2 and the lower tray assembly 3, meaning the materials used for the upper moldable component 21 and the upper bracket 22 are different. Moreover, the temperature required to heat the upper moldable component and the lower moldable component to deform is different from that required for the upper bracket and the lower bracket. The upper bracket 22 and the lower bracket 32 require materials that are more stable compared to the upper moldable component 21 and the lower moldable component 31, possessing high thermal stability to maintain their original shape after molding and provide structural support for the oral appliance 1. The upper moldable component 21 and the lower moldable component 31 are made from pliable materials, and the temperature required to heat the upper moldable component 21 and the lower moldable component 31 to deform is less than 100 degrees Celsius. The upper bracket 22 and the lower bracket 32 are made from materials with high thermal stability, and the temperature required to heat the upper bracket 22 and the lower bracket 32 to deform is greater than 100 degrees Celsius. In some cases, the upper tray assembly 2 and the upper tray assembly 3 also utilize a single material, specifically processed to achieve varying degrees of material rigidity. The hardness of the materials for the upper bracket 22 and the lower bracket 32 ranges between Shore D45 and Shore D95. When selecting materials for the oral appliance 1, considerations such as biocompatibility, durability, functional stability, patient comfort, and ease of cleaning are extensively evaluated. Suitable materials for oral medical devices that can be molded include plastics or polymers. These materials include, but are not limited to, polypropylene (PP), polycarbonate (PC), polyethylene terephthalate (PET), polymethyl methacrylate (PMMA), polysulfone (PPSU), polyethylene (PE), high-density polyethylene (HDPE), low-density polyethylene (LDPE), ethylene-vinyl acetate copolymer (EVA), thermoplastic polyurethane (TPU), styrene-ethylene-butylene-styrene (SEBS), styrene-ethylene-propylene-styrene (SEPS), and silicone rubber.

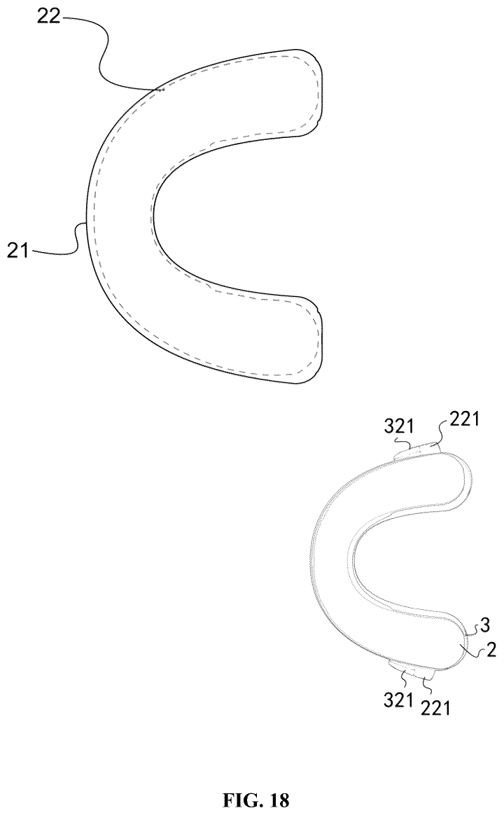

In this disclosure, the dimensions of the upper moldable component 21 and the lower moldable component 31, as well as the upper bracket 22 and the lower bracket 32, are specifically defined. The design features the upper moldable component 21 and the lower moldable component 31 having a larger projected area on a horizontal plane than that of the upper bracket 22 and the lower bracket 32 (as shown in FIG. 18), to provide greater comfort and adjustability. First, the upper moldable component 21 and the lower moldable component 31 are made from materials that are more flexible than those of the upper bracket 22 and the lower bracket 32, which helps to reduce irritation and abrasion to the soft tissues in the mouth. Secondly, considering the variation in the distance from the inside of the lips to the rear molar area among different patients, an oral appliance 1 that is too long might cause abrasions to the tissues in the molar area, particularly in areas near the joints, which could affect masticatory function. For individuals for whom the size of the upper moldable component 21 and the lower moldable component 31 is not suitable, it is possible to trim and adjust the length according to personal needs and preferences, thereby enhancing comfort and personal adaptability. This design not only ensures the functionality of the oral appliance 1 but also grants patients greater autonomy in making adjustments. Additionally, following this principle, since the upper and lower dental arches in the mouth are higher in the middle and lower on both sides, the upper tray assembly 2 and the upper tray assembly 3 are similarly designed to be higher in the middle and lower on both sides to prevent irritation and abrasion to the soft tissues in the mouth.

The following specific embodiments illustrate several configurations of the oral appliance 1 according to this disclosure.

### Embodiment 1

In this embodiment, the oral appliance 1 comprises: an upper tray assembly 2, including an upper moldable component 21 and an upper bracket 22, both configured with an arcuate outer shape to conform to the curvature of the patient's upper dental arch; and a lower tray assembly 3, including a lower moldable component 31 and a lower bracket 32, also configured with an arcuate outer shape to conform to the curvature of the patient's lower dental arch. The upper bracket 22 and the lower bracket 32 each include an outer wall 12 and a bottom wall 13, with sides positioned near the cheeks when worn, and the oral appliance has an end point 15 positioned away from the lip side. The upper moldable component 21 and the lower moldable component 31 are configured to engage with at least the bottom walls 13 of the upper bracket 22 and the lower bracket 32, respectively, and the temperature required to deform the upper moldable component 21 and the lower moldable component 31 is different from that of the upper bracket 22 and the lower bracket 32.

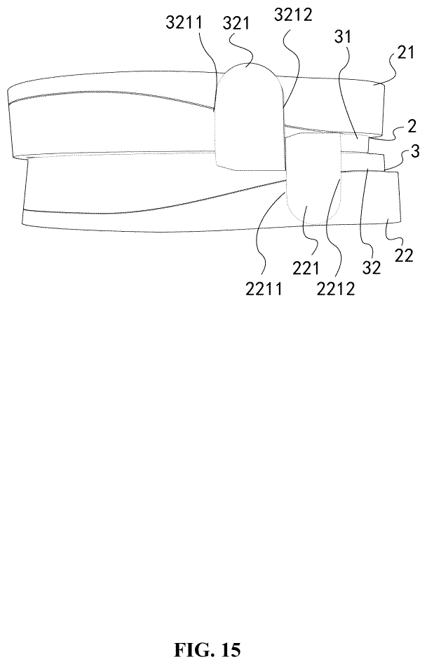

Furthermore, the upper bracket 22 and the lower bracket 23 each have at least one side of the two opposite sides near the cheeks that includes an upper protrusion 221 and a lower protrusion 321, respectively, and the upper protrusion 221 and the lower protrusion 321 are configured to allow the patient's mouth to open and close while maintaining a fixed anterior-posterior positional relationship between the upper bracket and the lower bracket when in use. The upper protrusion 221 has a first side 2211 closer to the end point 15 and an opposite second side 2212, while the lower protrusion 321 has a third side 3211 closer to the end point 15 and an opposite fourth side 3212. When worn, the second side 2212 contacts the third side 3211, thereby limiting the backward movement of the lower bracket 32 (as shown in FIG. 15).

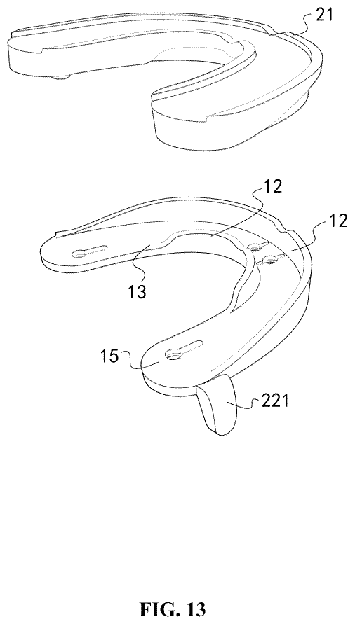

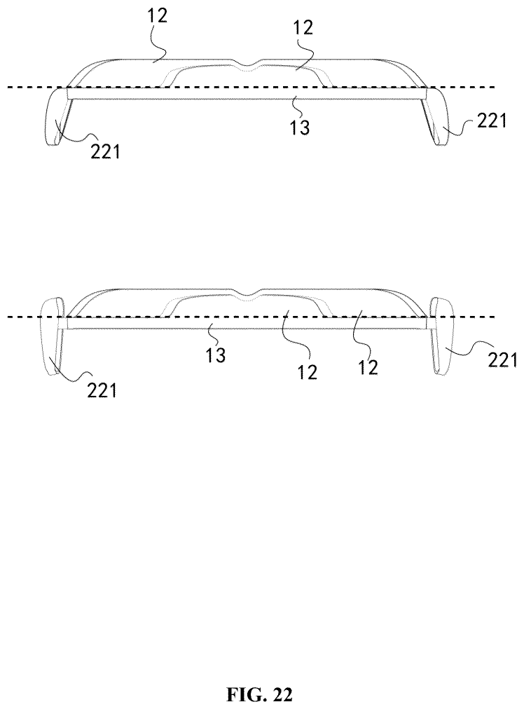

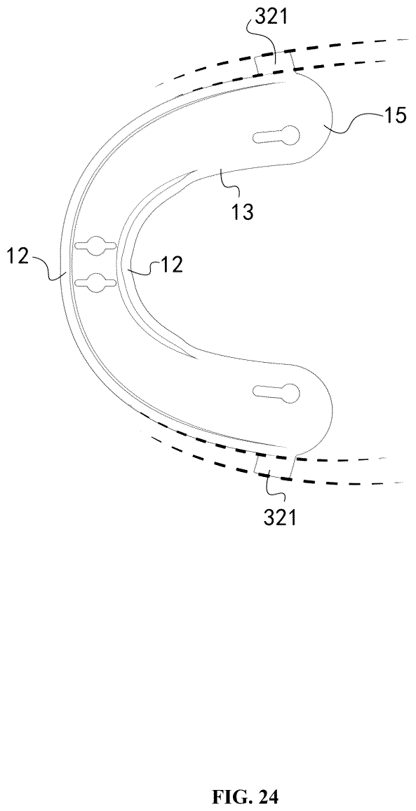

Specifically, the upper moldable component 21 and the lower moldable component 31 are configured to connect with the upper bracket 22 and the lower bracket 32, respectively, with the connection types including snap-fit, adhesive bonding, or groove connection. In this embodiment, the upper moldable component 21 and the lower moldable component 31 are connected to the upper bracket 22 and the lower bracket 32 via groove connections formed by the outer walls 12 and the bottom walls 13 (as shown in FIG. 13). This means that the outer walls 12 and the bottom walls 13 of the upper bracket 22 and the lower bracket 32 surround the upper moldable component 21 and the lower moldable component 31, holding them in place with two wall faces near the lip side and the tongue side. The connection between the upper bracket 22 and the lower bracket 32 replaces the traditional snap-fit structure, which is usually located on the bottom walls 13 of the upper and lower brackets, with upper protrusions and lower protrusions. In this embodiment, these upper and lower protrusions and the fixed snap-fit structure are identical and non-adjustable. The upper and lower protrusions can have various forms; in this case, they are configured in an L-shape, where the upper protrusion 221 of the upper bracket 22 and the lower protrusion 321 of the lower bracket 32 extend from the connection points with their respective brackets to form a complete upper protrusion 221 and a lower protrusion 321. This design reduces the volume of the oral appliance 1, enhances the comfort for the patient wearing it, and minimizes the risk of the upper and lower protrusions scraping the patient's oral cavity. Similarly, to reduce the volume of the oral appliance 1 and avoid scraping against the patient's oral cavity, the upper protrusion 221 and the lower protrusion 321 are shaped to conform to the curvature of the outer walls 12 of the upper bracket 22 and the lower bracket 32 (as shown in FIG. 24). In some instances, the upper protrusion 221 and the lower protrusion 321 align flush with the bottom walls 13 of the upper bracket 22 and the lower bracket 32 (as shown in FIG. 22), which can increase the stability of the protrusions. In other cases, it is also permissible for the upper protrusion 221 and the lower protrusion 321 to connect to the outer walls 12 of the upper bracket 22 and the lower bracket 32, respectively.

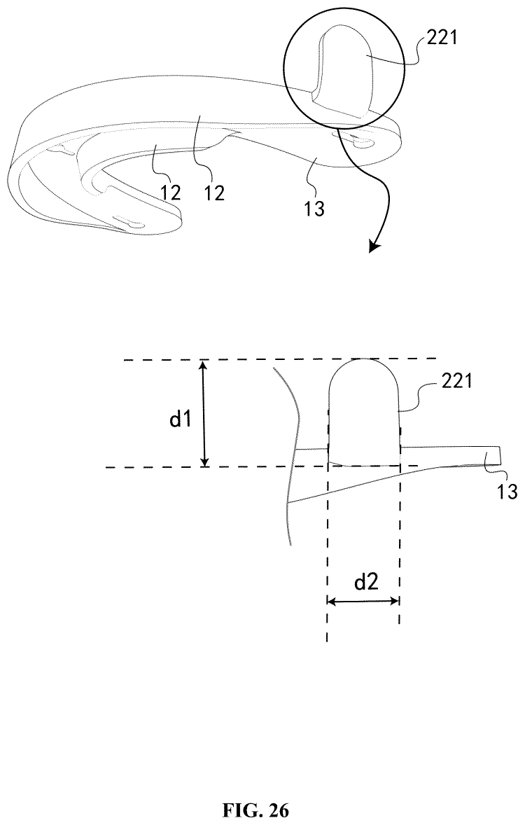

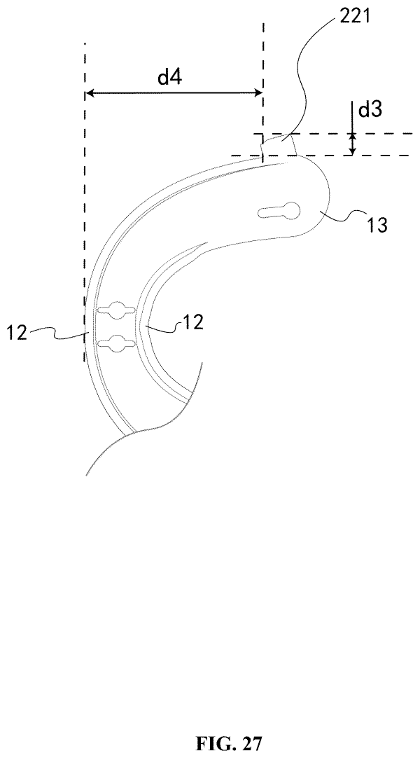

In this embodiment, the upper tray assembly 2 and the lower tray assembly 3 maintain the mandibular advancement function both when closed and separated, through the interaction/limitation between the upper protrusion 221 and the lower protrusion 321. The upper protrusion 221 is positioned behind the lower protrusion 321 to counteract the force exerted by the patient's mandible that tends to move the lower tray assembly 3 backward. As a result, the distance from the upper protrusion 221 to the end point 15 of the oral appliance is closer than that from the lower protrusion 321 to the end point 15. Additionally, at least a part of the upper protrusion 221 is set at a distance from the outer wall 12 of the upper bracket 22 that is greater than or equal to the distance from the lower protrusion 321 to the outer wall 12 of the lower bracket 32. The upper protrusion 221 and the lower protrusion 321 can be positioned anywhere on the upper bracket 22 and the lower bracket 32. Generally, to allow for a larger opening angle between the upper tray assembly 2 and the lower tray assembly 3 with shorter upper and lower protrusions, the upper protrusion 221 and the lower protrusion 321 are placed near the end point 15 of the upper bracket 22 and the lower bracket 32, respectively, away from the lip side. Moreover, when the upper protrusion 221 and the lower protrusion 321 are located on one of the outer walls 12 near the lip side, at least a part of them is positioned closer to the cheek than the outer walls 12, setting the distance between the upper protrusion 221, the lower protrusion 321, and the outer walls 12 to be less than 17.5 mm (as shown in FIG. 27, where d3≤17.5 mm). The upper protrusion 221 and the lower protrusion 321 have a specific length to ensure that when the upper tray assembly 2 and the lower tray assembly 3 are in an open state, the upper bracket 22 and the lower bracket 32 can contact and secure each other via the protrusions. The height range of the upper protrusion 221 and the lower protrusion 321 is 1.7 mm to 22.5 mm (as shown in FIG. 26, where 1.7 mm≤d1≤22.5 mm), to achieve a specific range of opening angles for the upper tray assembly 2 and the lower tray assembly 3, while minimizing impact on the patient's oral cavity. The upper bracket and the lower bracket each have a front end close to the lip side when worn, and the distance from the upper or lower protrusions to the respective front end is greater than 10 mm. The width of the upper protrusion 221 and the lower protrusion 321 also significantly affects the patient's oral cavity; hence, the width of the upper protrusion 221 and the lower protrusion 321 is set to be greater than 2.5 mm (as shown in FIG. 26, where d2≥2.5 mm). In some cases, the upper protrusion 221 and the lower protrusion 321 form an angle with the outer walls 12 (as shown in FIG. 22) and may also be parallel to the outer walls 12.

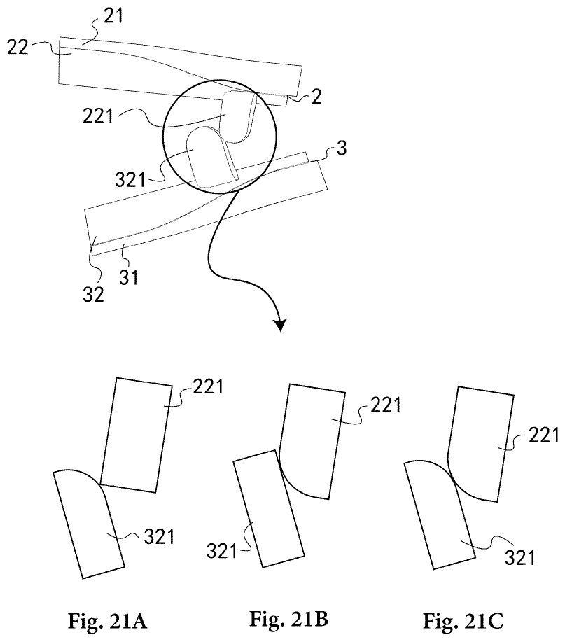

The upper protrusion 221 connects to the outer wall 12 of the upper bracket 22 and extends downward, while the lower protrusion 321 connects to the outer wall 12 of the lower bracket 32 and extends upward. When the oral appliance 1 is positioned in the patient's mouth, the second side 2212 of the upper protrusion 221 always contacts the third side 3211 of the lower protrusion 321. The contact points of the two protrusions during wear form at least one curvature (as shown in FIGS. 21A, 21B, and 21C). This curvature may be on one or both sides of the upper protrusion on the upper bracket, one or both sides of the lower protrusion on the lower bracket, or on both brackets. The curvature is crucial as the upper tray assembly 2 and the lower tray assembly 3 transition from a closed to an increasingly open position. The design of the curvature ensures a smooth transition during the opening and closing process of the oral appliance 1, reducing friction and resistance, and preventing structural sticking, thus providing a more stable and comfortable user experience and ensuring the continuity and effectiveness of the treatment.

The upper protrusion 221 and the lower protrusion 321 are typically integrally formed with the upper bracket 22 and the lower bracket 32, respectively, and made from at least one of the same materials as their respective brackets. In some cases, the upper protrusion 221 and the lower protrusion 321 may be non-integrally formed with the upper bracket 22 and the lower bracket 32, and can be made from different materials than the upper bracket 22 and the lower bracket 32.

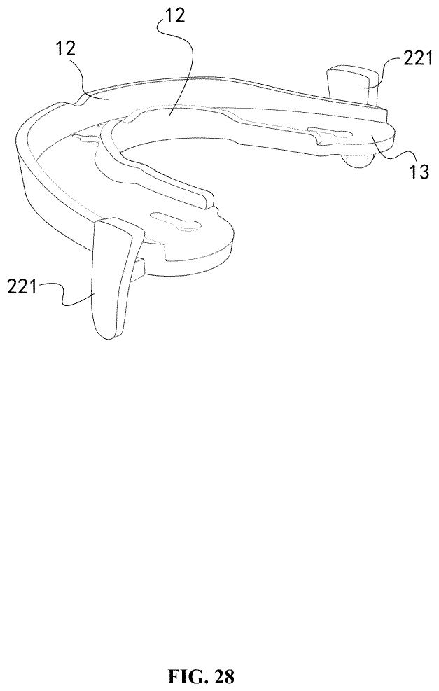

In other implementations, the protrusions may have shapes other than L-shape, such as T-shape or H-shape (as described in FIG. 28, where only the T-shaped protrusion is shown).

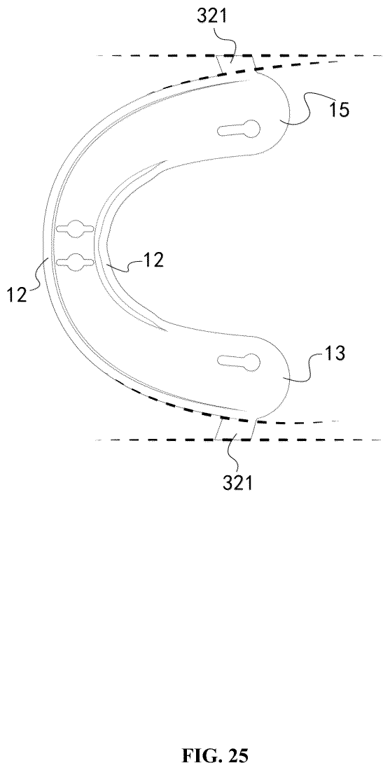

In other implementations, the curvature of the shape of the upper protrusion 221 and/or the lower protrusion 321 may differ from the curvature of the upper bracket 22 and the lower bracket 32 at the location of the upper protrusion 2211 and the lower protrusion 321 (as shown in FIG. 25).

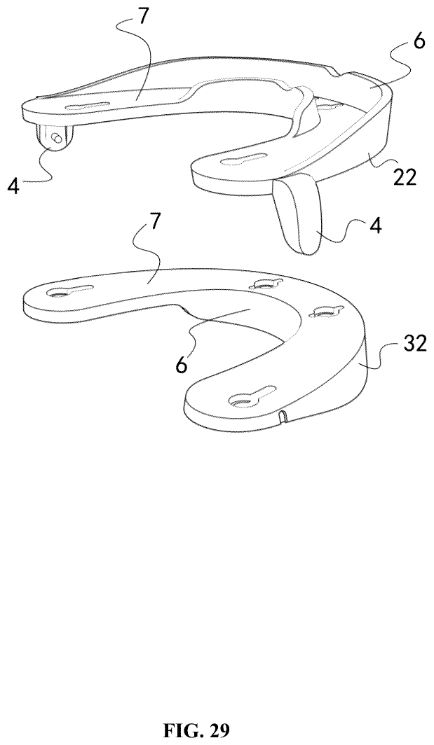

In yet other implementations, the upper protrusion 221 and the lower protrusion 321 may take different forms; for example, one of the upper bracket 22 or the lower bracket 32 may have an upper protrusion 221 or a lower protrusion 321 (as shown in FIG. 29).

Furthermore, the technical features described in the various embodiments above may be combined as needed to produce an oral appliance 1 that includes all or some of these features.

The oral appliance 1 implemented by this disclosure offers several beneficial effects:
1) Oral appliances on the market are primarily divided into two categories: customized and non-customized. Customized oral appliances are used to fit a patient's teeth through scanning, ensuring a perfect match with the patient's dental and oral conditions. This customization provides better comfort and stability, allowing the appliance to remain stable even when the mouth is open. However, they require customization under professional medical guidance and the use of high-precision and digital equipment, making them more expensive. Typically, a set of customized mandibular advancement devices can cost patients thousands of dollars and involve complex and cumbersome customization processes that require ongoing adjustments and coordination with a physician. On the other hand, non-customized oral appliances do not require dental scanning for customization, making them a more economical treatment option. They are also relatively easy to use; they simply need to be heated and molded to fit the upper and lower dental arches. However, because they are self-molded by the patient after heating, their fit and stability are not as good as those of customized appliances. Furthermore, user feedback collected from various e-commerce platforms indicates generally poor reviews for existing non-customized mandibular advancement devices. Based on the conclusions above, this disclosure innovatively combines the advantages of both customized and non-customized oral compliances, enhancing stability, therapeutic effectiveness, and user comfort while maintaining economic benefits. The oral appliance 1 is designed to remain effective even when the mouth opens and closes. This new oral appliance 1 offers both economic and functional advantages, providing patients with a better option.
2) The oral appliance 1 with upper and lower protrusions offers functional advantages. Traditional moldable oral appliances are fixed structures that limit oral movement when worn. In contrast, this disclosure's appliance with upper and lower protrusions allows patients to speak, drink, and perform other daily activities while wearing it, greatly expanding its range of application. This design ensures that patients do not need to frequently remove the appliance during treatment, enhancing user convenience. This convenience is particularly important for patients who need to wear oral appliances for extended periods, significantly improving their quality of life. Specifically, for some patients who experience throat snoring symptoms, these individuals may need to breathe through their mouths while sleeping, a situation that traditional moldable oral appliances struggle to accommodate. The protrusion design of this oral appliance 1 can adapt to various oral movements, thus offering more effective treatment for a broader patient group. This multifunctional design meets the personalized needs of different patients.
3) The design and development of the oral appliance 1 with upper and lower protrusions is not just about structure; it involves a complex and rigorous process that incorporates multiple types of data for design, testing, and experimentation. Initially, it requires gathering oral data from different patient groups to ensure that the device meets the needs of various oral structures. Subsequently, the collected data is used for preliminary design and simulation, material selection and testing, numerous experimental tests and optimizations, and population trials and validations to ensure the appliance's functionality and comfort under diverse conditions. Finally, the optimal structure of the oral appliance 1 is determined, including the form and placement of the upper and lower protrusions, material thickness, and the angles, height, and length of the structure. Each step is meticulously calculated and repeatedly validated, ensuring the product achieves the best possible functionality, comfort, and safety, while providing an effective and comfortable treatment experience for patients.
4) Dentistry is a precise and data-driven discipline where even minor variations in data (such as angles, weight, dimensions, and the softness of materials) can impact patient comfort and the effectiveness of use. This disclosure meticulously adjusts and repeatedly tests these variables to achieve optimal usage and comfort levels. Further in-depth research into the design of oral appliances 1 has addressed numerous challenges, enhancing comfort during treatment. Since oral appliances must conform closely to the human body, they require extensive consideration of human factors engineering and are designed and tested using substantial human data, making them complex and challenging technologies when designed and manufactured. Specifically, during the process of opening the mouth to wear the appliance, it must counteract its own weight to maintain stable engagement with the upper dental arch of the patient; the heights and widths of the upper and lower protrusions are constrained to allow patients to adapt to the prescribed opening angles while minimizing discomfort when worn; and the design of the upper and lower protrusions ensures that they consistently restrict the lower jaw during mouth opening and closing, maintaining the connection between the upper bracket 22 and the lower bracket 32. Solutions to these issues have been meticulously designed and extensively tested. This includes optimizing the details of the upper bracket 22 and the lower bracket 32 and the upper moldable component 21 and lower moldable component 31 to better fit the upper and lower dental arches. In particular, the upper tray assembly 2 is closely matched to the upper dental arch to counteract gravity when the mouth is open, ensuring stability; the height range of the upper and lower protrusions is set between 1.7 mm and 22.5 mm to allow for a greater range of mouth opening and closing angles of the oral appliance 1 without conflicting with the patient's oral structure. The distances from the upper and lower protrusions to the front end are set to be greater than 10 mm. While positioning the upper and lower protrusions closer to the end point increases the opening angle between the upper bracket 22 and the lower bracket 32, it can also lead to more frequent contact with the patient's oral structures, potentially causing a foreign body sensation. Therefore, finding a balance between these factors is essential. The upper bracket 22 and the lower bracket 32 need the upper and lower protrusions to engage at any angle within a certain range, so positioning the upper and lower protrusions outward at a certain distance is crucial to ensure therapeutic effectiveness and enhance patient comfort. Furthermore, it's important to design at least one curvature at the contact points of the upper and lower protrusions of the upper and lower brackets. This curvature ensures smooth operation of the brackets during opening and closing, reducing friction and the possibility of getting stuck. These designs are not merely simple structural forms but result from precise calculations and optimizations. To achieve the best outcomes, numerous experimental tests and data recordings are necessary. Through repeated trials and minor adjustments, the shapes and metrics of the upper and lower protrusions are gradually refined. Extensive data analysis and feedback are used to optimize the design concerning the upper and lower protrusions and other aspects, ensuring that every detail undergoes rigorous verification and optimization. This meticulous approach guarantees the comfort of the patients during use and the durability of the oral appliance.
5) The oral appliance 1 of this disclosure also has the following advantages: a. The oral appliance 1 with upper and lower protrusions enhances treatment stability. The structure of the upper and lower protrusions provides a reliable fixation mechanism, reducing movement of the oral appliance 1 and the risk of dislodgement within the oral cavity. This ensures that the oral appliance 1 maintains its effective therapeutic impact in various jaw positions, minimizing the risk of treatment interruptions and improving both the effectiveness and stability of the product. Additionally, the enhanced stability of this disclosure's oral appliance 1 means that patients do not need to replace it frequently, reducing subsequent replacement costs. The fixed structure of the upper and lower protrusions requires patients to purchase a set of upper and lower tray assemblies 2 and 3 to ensure the appropriate mandibular advancement distance. While this increases the initial cost to the patient, the fixed protrusions are more stable and durable compared to adjustable structures, reducing abandonment due to damage and further lowering the overall treatment costs. b. Unlike conventional customized mandibular advancement devices, whose main body (i.e., the part that directly contacts the teeth) is typically made from a rigid material, this disclosure's oral device features flexible materials that contact the inner mouth. These materials do not irritate the patient's oral tissues, reducing the foreign body sensation when wearing the product. Additionally, in the event of an accidental impact, the flexible materials act as a cushion, protecting the teeth and offering a safer oral device. c. This disclosure's oral device can be quickly produced and used, saving patients the waiting time associated with purchasing customized devices. This makes it a superior option for patients seeking prompt treatment.

It must be noted that as used herein and in the appended claims, the regular forms “a” “an” “the” include their plural equivalents, unless the context clearly dictates otherwise.

1. An oral device with protrusions, comprising:
an upper tray assembly, including an upper moldable component and an upper bracket, and having an arcuate outer shape configured to conform to a curvature of a patient's upper dental arch;
a lower tray assembly, including a lower moldable component and a lower bracket, and having an arcuate outer shape configured to conform to a curvature of the patient's lower dental arch;
wherein the upper bracket and the lower bracket each are formed by an outer wall and a bottom wall, each having two opposite sides configured to be near cheeks when in use, wherein the oral device has an end point configured to be positioned away from a lip side, wherein the upper moldable component is configured to be in connection with at least the bottom wall of the upper bracket, wherein the lower moldable component is configured to be in connection with at least the bottom wall of the lower bracket, and wherein a temperature required to heat the upper moldable component and the lower moldable component to deform is different from that required for the upper bracket and the lower bracket;
wherein the upper bracket and the lower bracket each have at least one side of the two opposite sides configured to be near the cheeks that includes an upper protrusion and a lower protrusion, respectively, and wherein the upper protrusion and the lower protrusion are configured to allow the patient's mouth to open and close while maintaining a fixed anterior-posterior positional relationship between the upper bracket and the lower bracket when in use;
wherein the upper protrusion has a first side nearer the end point and a second side opposite the first side, wherein the lower protrusion has a third side nearer the end point and a fourth side opposite the third side, and wherein the second side and the third side are configured to be in contact when in use to limit a distance of an anterior displacement of the lower bracket;
wherein the upper protrusion and the lower protrusion each have at least a portion configured to be positioned nearer the cheeks than the outer walls of the upper bracket and the lower bracket;
wherein a distance from the upper protrusion to the end point of the oral device is shorter than a distance from the lower protrusion to the end point of the oral device;
wherein the upper protrusion extends in a first direction past the bottom wall of the upper bracket toward the lower bracket to overlap the lower bracket in a second direction and the lower protrusion extends in the first direction past the bottom wall of the lower bracket towards the upper bracket and overlaps the upper bracket in the second direction,
wherein a first side and a second side of each of the upper protrusion and lower protrusion extend outwardly in the second direction from the outer walls of the upper bracket and lower bracket, respectively, and an inner wall and an outer wall extend between the first side and the second side,
wherein the upper tray assembly and lower tray assembly are stacked in a vertical direction, and
wherein the upper protrusion and the lower protrusion form an angle with an axis extending in the vertical direction.

2. The oral device according to claim 1, wherein the arcuate outer shapes of the upper tray assembly and the lower tray assembly adopt at least one of an ovoid arch, a tapered arch, or a square arch.

3. The oral device according to claim 1, wherein a material of the upper moldable component and the lower moldable component includes a flexible thermoplastic material.

4. The oral device according to claim 3, wherein the upper moldable component and the lower moldable component are configured to soften when heated in hot water, and to allow them to be adjusted and molded to match the curvatures of the patient's upper dental arch and lower dental arch during biting.

5. The oral device according to claim 1, wherein a hardness of a material of the upper bracket and the lower bracket is at least partially greater than a hardness of a material of the upper moldable component and the lower moldable component.

6. The oral device according to claim 1, wherein the connection between the upper moldable component and the bottom wall of the upper bracket, and between the lower moldable component and the bottom wall of the lower bracket, is established through one of the following methods: snap-fit connection, adhesive bonding, or groove connection.

7. An oral device with protrusions, comprising:
an upper tray assembly, including an upper moldable component and an upper bracket, configured to be positioned adjacent to a patient's upper dental arch when configured to be placed inside the patient's mouth;
a lower tray assembly, including a lower moldable component and a lower bracket, configured to be positioned adjacent to the patient's lower dental arch when configured to be placed inside the patient's mouth;
wherein the upper bracket and the lower bracket each are formed by an outer wall and a bottom wall, each having two opposite sides configured to be near cheeks when in use, wherein the oral device has an end point configured to be positioned away from a lip side, wherein the upper moldable component is configured to be in connection with at least the bottom wall of the upper bracket, wherein the lower moldable component is configured to be in connection with at least the bottom wall of the lower bracket, and wherein a temperature required to heat the upper moldable component and the lower moldable component to deform is different from that required for the upper bracket and the lower bracket;
wherein the upper bracket and the lower bracket each have at least one side of the two opposite sides configured to be near the cheeks that includes an upper protrusion and a lower protrusion, respectively, and wherein the upper protrusion and the lower protrusion are configured to allow the patient's mouth to open and close while maintaining a fixed anterior-posterior positional relationship between the upper bracket and the lower bracket when in use;
wherein the upper protrusion has a first side nearer the end point and a second side opposite the first side, and the lower protrusion has a third side nearer the end point and a fourth side opposite the third side, wherein the second side and the third side are configured to be in contact when in use to limit a distance of an anterior displacement of the lower bracket, wherein the second side and/or the third side is curved;
wherein a distance from the upper protrusion to the end point of the oral device is shorter than a distance from the lower protrusion to the end point of the oral device;
wherein the upper protrusion extends in a first direction past the bottom wall of the upper bracket toward the lower bracket to overlap the lower bracket in a second direction and the lower protrusion extends in the first direction past the bottom wall of the lower bracket towards the upper bracket and overlaps the upper bracket in the second direction,
wherein the upper tray assembly and lower tray assembly are stacked in a vertical direction,
wherein the upper protrusion and the lower protrusion form an angle with an axis extending in the vertical direction, and
wherein the upper protrusion is immovable relative to the upper bracket, and the lower protrusion is immovable relative to the lower bracket.

8. The oral device according to claim 7, wherein a distance between at least a portion of the upper protrusion and the outer wall of the upper bracket is greater than or equal to a distance between the lower protrusion and the outer wall of the lower bracket.

9. The oral device according to claim 7, wherein the connection between the upper moldable component and the upper bracket, and between the lower moldable component and the lower bracket, is established through grooves formed by the outer walls and the bottom walls of the upper bracket and the lower bracket, respectively.

10. The oral device according to claim 7, wherein the upper protrusion and the lower protrusion have shapes that conform to curvatures of the outer walls of the upper bracket and the lower bracket, respectively.

11. The oral device according to claim 7, wherein the upper moldable component and the lower moldable component include a material with plasticity, and the temperature required to heat the upper moldable component and the lower moldable component to deform is less than 100 degrees Celsius.

12. The oral device according to claim 11, wherein the upper bracket and the lower bracket include a material with high thermal stability, and the temperature required to heat the upper bracket and the lower bracket to deform is greater than 100 degrees Celsius.

13. An oral device with protrusions, comprising:
an upper tray assembly, including an upper moldable component and an upper bracket, and having an arcuate outer shape configured to conform to a curvature of a patient's upper dental arch;
a lower tray assembly, including a lower moldable component and a lower bracket, and having an arcuate outer shape configured to conform to a curvature of the patient's lower dental arch;
wherein the upper bracket and the lower bracket each are formed by an outer wall and a bottom wall, each having two opposite sides configured to be near cheeks when in use, wherein the oral device has an end point configured to be positioned away from a lip side, wherein the upper moldable component is configured to be in connection with at least the bottom wall of the upper bracket, wherein the lower moldable component is configured to be in connection with at least the bottom wall of the lower bracket, and wherein a temperature required to heat the upper moldable component and the lower moldable component to deform is different from that required for the upper bracket and the lower bracket;
wherein the upper bracket and the lower bracket each have at least one side of the two opposite sides configured to be near the cheeks that includes an upper protrusion and a lower protrusion, respectively, and wherein the upper protrusion and the lower protrusion are configured to allow the patient's mouth to open and close while maintaining a fixed anterior-posterior positional relationship between the upper bracket and the lower bracket when in use;
wherein the upper protrusion has a first side nearer the end point and a second side opposite the first side, and the lower protrusion has a third side nearer the end point and a fourth side opposite the third side, wherein the second side and the third side are configured to be in contact when in use to limit a distance of an anterior displacement of the lower bracket;
wherein the upper protrusion and the lower protrusion have one or more of the following characteristics:
a. a height of the upper protrusion and the lower protrusion being between 1.7 mm to 22.5 mm;
b. a distance from the upper protrusion and the lower protrusion to the outer walls of the upper bracket and the lower bracket, respectively, being less than 17.5 mm;
wherein the upper protrusion extends in a first direction past the bottom wall of the upper bracket toward the lower bracket to overlap the lower bracket in a second direction and the lower protrusion extends in the first direction past the bottom wall of the lower bracket towards the upper bracket and overlaps the upper bracket in the second direction,
wherein the upper protrusion and lower protrusion extend outwardly in the second direction from the outer walls of the upper bracket and lower bracket, respectively,
wherein the upper tray assembly and lower tray assembly are stacked in a vertical direction,
wherein the upper protrusion and the lower protrusion form an angle with an axis extending in the vertical direction, and
wherein the upper protrusion is immovable relative to the upper bracket, and the lower protrusion is immovable relative to the lower bracket.

14. The oral device according to claim 13, wherein the upper tray assembly and the lower tray assembly are shaped such that a middle is higher than both sides.

15. The oral device according to claim 13, wherein a projected area of the upper moldable component and the lower moldable component on a horizontal plane is greater than a projected area of the upper bracket and the lower bracket on the same plane.

16. The oral device according to claim 13, wherein the outer walls of the upper bracket and the lower bracket have a continuous curvature.

17. The oral device according to claim 15, wherein a connection point of the upper protrusion and the upper bracket, and/or of the lower protrusion and the lower bracket, is flush with the bottom walls of the upper bracket and the lower bracket.

18. An oral device with protrusions, comprising:
an upper tray assembly, including an upper moldable component and an upper bracket, configured to be positioned adjacent to a patient's upper dental arch when configured to be placed inside the patient's mouth;
a lower tray assembly, including a lower moldable component and a lower bracket, configured to be positioned adjacent to the patient's lower dental arch when configured to be placed inside the patient's mouth;
wherein the upper bracket and the lower bracket each are formed by an outer wall and a bottom wall, each having two opposite sides configured to be near cheeks when in use, wherein the oral device has an end point configured to be positioned away from a lip side, wherein the upper moldable component is configured to be in connection with at least the bottom wall of the upper bracket, wherein the lower moldable component is configured to be in connection with at least the bottom wall of the lower bracket, and wherein a temperature required to heat the upper moldable component and the lower moldable component to deform is different from that required for the upper bracket and the lower bracket;
wherein the upper bracket and the lower bracket each have at least one side of the two opposite sides configured to be near the cheeks that includes an upper protrusion and a lower protrusion, respectively, and wherein the upper protrusion and the lower protrusion are configured to allow the patient's mouth to open and close while maintaining a fixed anterior-posterior positional relationship between the upper bracket and the lower bracket when in use;
wherein the upper protrusion has a first side nearer the end point and a second side opposite the first side, and the lower protrusion has a third side nearer the end point and a fourth side opposite the third side, wherein the second side and the third side are configured to be in contact when in use to limit a distance of an anterior displacement of the lower bracket;
wherein the upper bracket and the lower bracket have a front end near the lip side when in use, and a distance from the upper protrusion and/or lower protrusion to the front end is greater than 10 mm;
wherein the upper protrusion extends in a first direction past the bottom wall of the upper bracket toward the lower bracket to overlap the lower bracket in a second direction and the lower protrusion extends in the first direction past the bottom wall of the lower bracket towards the upper bracket and overlaps the upper bracket in the second direction,
wherein a first side and a second side of each of the upper protrusion and lower protrusion extend outwardly in the second direction from the outer walls of the upper bracket and lower bracket, respectively, and an inner wall and an outer wall extend between the first side and the second side,
wherein the upper tray assembly and lower tray assembly are stacked in a vertical direction, and
wherein the upper protrusion and the lower protrusion form an angle with an axis extending in the vertical direction.

19. The oral device according to claim 18, wherein a width of the upper protrusion and the lower protrusion is greater than 2.5 mm.

20. The oral device according to claim 18, wherein the upper tray assembly and the lower tray assembly include at least two different materials.

21. The oral device according to claim 18, wherein a projected area of the upper moldable component and the lower moldable component on a horizontal plane is greater than a projected area of the upper bracket and the lower bracket on the same plane.

22. (canceled)
